(19) United States
(12) Patent Application Publication
(10) Pub. No.: US 2008/0268459 A1
(43) Pub. Date: Oct. 30, 2008

(54) Anti-Lewis Y Anti-Idiotypic Antibodies and Uses Thereof

(75) Inventors: Zhanqi Liu, Melbourne (AU); Andrew Mark Scott, Kew (AU); Fiona E. Smyth, Victoria (AU)

Correspondence Address: FULBRIGHT & JAWORSKI, LLP, 666 FIFTH AVE, NEW YORK, NY, 10103-3198, US

(73) Assignee: Wyeth Ludwig Institute for Cancer

(21) Appl. No.: 10/568,255

(22) PCT Filed: Aug. 10, 2004
(86) PCT No.: PCT/US2004/025789

§ 371 Date: Jul. 8, 2008

Related U.S. Application Data
(60) Provisional application No. 60/495,557, filed on Aug. 14, 2003.

(51) Int. Cl. G01N 33/53; C07K 16/00; C12N 5/12

(52) U.S. Cl. 435/7.1; 530/387.2; 435/346

(57) ABSTRACT

This invention provides anti-idiotype antibodies specific for Anti-Lewis Y monoclonal antibodies. The present invention also directed against an ELISA screening method of mAbs produced by hybridoma clones for specific binding to the variable regions of hu3S193 and the ability of the anti-idiotypic mAB to inhibit hu3S193 binding to Lewis Y antigen. Additionally, the present invention provides a hybridoma capable of producing an anti-idiotype antibody specific for anti-Lewis Y monoclonal antibody. A further aspect of the invention is to provide a hybridoma, which is specific for anti-Lewis Y monoclonal antibody selected from the group consisting of LMH-1, LMH-2, LMH-3, or LMH-4. The present invention is also directed against a method to detect HAMA, HACA and HAHA responses using the antibody of the invention.

### FIELD OF THE INVENTION

The present invention is related to the field of molecular biology, and more particularly to antibodies.

### BACKGROUND

The advent of monoclonal antibody (mAb) technology 25 years ago has provided an enormous repertoire of useful research reagents and created the opportunity to use antibodies as approved pharmaceutical reagents in cancer therapy, autoimmune disorders, transplant rejection, antiviral prophylaxis and as anti-thrombotics (Glennie and Johnson 2000). The application of molecular engineering to convert murine mAbs into chimeric mAbs (mouse V-region, human C-region) and humanized reagents where only the mAb complementarity-determining regions (CDR) are of murine origin has been critical to the clinical success of mAb therapy. The engineered mAbs have markedly reduced or absent immunogenicity, increased serum half-life and the human Fc portion of the mAb increases the potential to recruit the immune effectors of complement and cytotoxic cells (Clark 2000). Investigations into the biodistribution, pharmacokinetics and any induction of an immune response to clinically administered mAbs requires the development of analyses to discriminate between the pharmaceutical and endogenous proteins.

An antibody is usually defined in terms of the antigen it recognizes. An antibody's specificity for a particular antigen is determined by its antigen-binding site, the distinct region of the antibody molecule that makes contact with an antigen. This site is found within the variable regions of immunoglobulin heavy and light chains. However, an antibody may also be defined by its idiotype, an ensemble of idiotopes, or surface markers, associated with the unique heavy and light chains regions of a monospecific population of antibody molecules. The antigenic determinants unique to an antibody are termed idiotopes and are defined by the reaction of anti-idiotopic antibodies with the antibody bearing the idiotopes. Idiotypes are useful markers because they enable researchers to follow the appearance and persistence of particular antibodies in immune responses and inherited immunoglobulin genes. Idiotypes are also unique determinants that can stimulate production of anti-idiotype antibodies.

Anti-idiotype antibodies bind the variable region of other antibodies and in the immune system play a major role in binding antibodies raised against internal (self) antigens (Jerne 1974). Anti-idiotype antibodies elicited by the variable region of the inducing idiotype antibody can be one of three categories: 1) recognizing an epitope in the antigen binding site (paratope); 2) binding near the paratope and sterically interfering with the idiotype antibody-antigen interaction; or 3) structurally mimicking the antigen recognized by the idiotype antibody (“internal image” anti-idiotype antibody). Anti-idiotypes of the latter category have been produced and investigated as vaccines given their potential in modulating hosts' immune responses to tumor-associated antigens in addition to bacterial, viral and parasitic infections (reviewed in (Bhattacharya-Chatterjee, Chatterjee et al. 2001). Rodent anti-idiotype antibodies require an adjuvant and conjugation to a carrier, usually keyhole limpet hemocyanin (KLH) for immune or anti-tumor responses to be elicited.

Anti-idiotype antibodies can also be utilized as enzyme linked immunosorbent assay, hereinafter referred to as ELISA, reagents in immunotherapy trials for detecting patient serum levels of injected idiotype antibodies or immune responses to administered idiotype antibodies. Such immune responses include human anti-mouse antibody, human anti-chimeric antibody, and human anti-humanized antibody (hereinafter referred to as HAMA, HACA, HAHA respectively). Anti-idiotype antibody NUH-82 is directed against the binding site of a murine mAb G250 which recognizes the G250 antigen on human renal cell carcinomas (Uemura, Okajima et al. 1994). NUH-82 has proven useful for measuring HACA responses in patients receiving cG250 antibody radioimmunotherapy (Steffens, Boerman et al. 1997) and more recently NUH-82 has been used in a sandwich ELISA to measure serum levels of cG250 in patient sera for pharmacokinetic analyses (Liu, Smyth et al. 2002).

The hu3S193 antibody was developed for targeting Lewis Y, a difucosylated carbohydrate antigen, expressing epithelial tumors including breast, colon, lung, prostate and ovarian carcinomas (Kitamura, Stockert et al. 1994; Scott, Geleick et al. 2000). Lewis Y is a carbohydrate molecule expressed on the surface of tumor cells of epithelial origin, e.g. lung, intestinal, breast, prostate or ovarian cancers. The Lewis Y antigen is expressed in normal tissues but the level of expression is higher in certain tumor types so that the antigen can be used as a marker for cells of some breast, colon, gastric, esophageal, pancreatic, duodenal, lung, bladder and renal carcinomas and gastric and islet cell neuroendocrine tumors. The Lewis Y associated-associated antigen is an attractive target for immunotherapy due to its high density and homogeneous expression in primary and metastatic lesions (Kim, Yuan et al. 1986; Sakamoto, Furukawa et al. 1986; Zhang, Cordon-Cardo et al. 1997).

Hu3S193 elicits strong ADCC and CDC in vitro, has shown promise as an immunotherapeutic of selected solid tumors in preclinical studies (Clarke, Lee et al. 2000b; Clarke, Lee et al. 2000a), and is currently in Phase I dose escalation clinical trials. Preclinical biodistribution studies traced radiolabel led hu3S193 to evaluate the tumor targeting capabilities of the stable radioconjugate in an animal model of breast cancer (Clarke, Lee et al. 2000a). In the clinic, mAbs such as hu3S193 can be traced using radiolabelling to evaluate biodistribution and tumor imaging, and the clearance of the radioconjugate from the serum, as a measure of the mAb's pharmacokinetics (Clarke, Lee et al. 2000a; Scott, Geleick et al. 2000; Hoffman, Scott et al. 2001). However, in the absence of clinical symptoms a HAHA immune response cannot be detected.

Given the immune effector functions of hu3S193, determining the serum levels of administered antibody in early trials would assist the planning of future therapeutic regimens and enable determination of unlabelled hu3S193 pharmacokinetics, while the ability to measure HAHA would assist the safety evaluation of hu3S193.

Specifically, the invention is also directed to the use of anti-idiotypes in immunotherapy trials as diagnostic reagents for monitoring the pharmacokinetics of the administered idiotype that they recognize in the circulation of patients. Similarly the anti-idiotype can be used as a positive control for HAHA, HACA or HAMA immune responses to the administered idiotype mAb. Such immune responses may have no influence on the clinical outcome some in patient populations (Gruber, van Haarlem et al. 2000), or can be associated with hypersensitive reactions and dramatic changes in pharmcokinetics and biodistribution of the immunotherapeutic mAb that preclude further treatment. (Clark 2000).

### BRIEF DESCRIPTION OF THE FIGURES

FIG. 1A-B: The binding specificity of the anti-hu3S193 monoclonal antibodies, LMH-1 (•), -2 (∇), -3 (⋄), and anti-hulgG-4 (⋄) were determined by ELISA. Serially diluted hybridoma culture supernatants were examined for binding activity to microtitre plates coated with A) hu3S193 (FIG. 1A) and B) hulgG (FIG. 1B). Controls for secondary conjugate (▴) and substrate alone (∘) were also included. Specific binding for hu3S193 was observed with LMH-1, -2 and -3. LMH-4 demonstrated anti-hulgG binding activity.

FIG. 2: After clonal expansion, the hybridoma culture supernatants were examined in triplicate by ELISA for the ability to neutralize hu3S193 antigen binding activity. Mean ±SD results demonstrated the antagonist activity of anti-hu3S193 mAbs, LMH-1, -2, and -3 with the blocking of hu3S193 binding to plates coated with synthetic Le^{y} antigen

FIG. 3A-D: Verification of LMH-2 and LMH-3 specificity for the 3S193 idiotope. FIG. 3A. Protein-A purified LMH-3 was coated on ELISA plates and serially diluted triplicate samples of 10 □g/ml hu3S193, mu3S193, BR55.2 and control IgG1 mAbs huA33 and muA33 were added to the wells to further characterize the binding specificity of LMH-3. An additional favorable feature of this clone was that biotinylated LMH-3 could be utilized for detection of bound 3S193 mAb captured with LMH-3. FIG. 3B. Antagonistic binding preventing binding to Le^{y}-tetrasaccaride-coated plates by anti-Le^{y} and anti-idiotype mAbs for hu3S193-Le^{y} antigen binding; FIG. 3C. mu3S193-Le^{y} antigen binding; and FIG. 3D. BR55.2-Le^{y} antigen binding activity.

FIG. 4A-C: Development of ELISA for measuring hu3S193 in patient sera samples. FIG. 4A. Method available prior to anti-idiotype availability—capture with synthetic antigen Le^{y}—BSA conjugate, detection with anti-hulgG demonstrates low sensitivity with hu3S193 standard and high background in patient pre-infusion sera samples. FIG. 4B. Capture with anti-hu3S193 idiotype LMH-2 and detection with LMH-2-biotin. Low sensitivity observed and FIG. 4C. Capture with anti-hu3S193 idiotype LMH-3 and detection with LMH-3-biotin demonstrates minimal background and sensitivity to 3 ng/ml hu3S193 in serum.

FIG. 5: Biacore analyses of serum HAHA responses to hu3S193 were optimized and validated using anti-idiotype LMH-3 as positive control. Hu3S193 was immobilized on the Biosensor chip and serial dilutions of anti-idiotype mAb LMH-3 (▪) and isotype-matched control antibody (▴) in human serum μg/ml) were passed over the chip in triplicate.

### SUMMARY OF THE INVENTION

The present invention is directed against an anti-idiotypic antibody against humanized anti-Lewis Y monoclonal antibody, hu3S193. The present invention also directed against an ELISA screening method of mAbs produced by hybridoma clones for specific binding to the variable regions of hu3S193 and the ability of the anti-idiotypic mAB to inhibit hu3S193 binding to Lewis Y antigen.

The present invention is also directed against a method to detect HAMA, HACA and HAHA responses using the antibody of the invention.

One aspect of the invention is to provide anti-idiotype antibodies specific for Anti-Lewis Y monoclonal antibody. Another aspect of the invention is to provide anti-idiotype antibodies, specific for the anti-Lewis Y antibody which binds to the variable region of an anti-Lewis Y monoclonal antibody. Similarly, another aspect of the invention is an anti-idiotype antibody which blocks the binding of an anti-Lewis Y monoclonal antibody. Another aspect of the invention is to provide an anti-idiotype antibody which specifically binds hu3S193. Specifically, the anti-idiotype antibodies provided for in this invention can be selected from the group consisting of a monoclonal antibody, a chimeric antibody, a human antibody, a humanized antibody, or a single chain antibody.

Another aspect of the invention is to provide a hybridoma capable of producing an anti-idiotype antibody specific for anti-Lewis Y monoclonal antibody. A further aspect of the invention is to provide a hybridoma which is specific for anti-Lewis Y monoclonal antibody selected from the group consisting of LMH-1, LMH-2, and LMH-3.

Another aspect of the invention is to provide methods for detecting the ability of an anti-idiotype antibody to inhibit the binding of antibody to antigen. Another aspect of the invention is to provide methods for detecting the ability of anti-idiotype to capture and detect bound idiotype antibody. Another aspect of the invention is to provide methods for detecting the ability of anti-idiotype antibody to bind to idiotype anti-Lewis Y antibody. Another aspect of the invention are methods of detecting the amount of antibody in sample serum.

The present invention includes methods of measuring hu3S193 in sera samples by ELISA using synthetic Lewis Y tetrasaccharide coupled to BSA as antigen to capture hu3S193 and commercially available anti-human antibody preparations as detectors of bound hu3S193. The sensitivity of the assay has been greatly restricted by the non-specific binding of hulgG from the sera samples being detected by the secondary anti-hulgG antibody resulting in high background. Based upon experiences with measuring HACA responses to cG250 and serum levels of cG250 with anti-idiotype NUH-82, the production of a suitable anti-idiotype hu3S193 mAb became a necessity.

The present invention is also directed against a method to detect HAMA, HACA and HAHA responses using the antibody of the invention

### DETAILED DESCRIPTION OF THE INVENTION

This invention provides anti-idiotype antibodies specific for anti-Lewis Y monoclonal antibodies. These anti-idiotype antibodies are specific for the anti-Lewis Y antibody, which binds to the variable region of an anti-Lewis Y monoclonal antibody. Similarly, another aspect of the invention is an anti-idiotype antibody, which blocks the binding of an anti-Lewis Y monoclonal antibody. Another aspect of the invention is to provide an anti-idiotype antibody, which specifically binds the anti-Lewis Y monoclonal, hu3S193. Specifically, the anti-idiotype antibodies provided for in this invention can be selected from the group consisting of a monoclonal antibody, a chimeric antibody, a human antibody, a humanized antibody, or a single chain antibody.

Additionally, the present invention provides a hybridoma capable of producing an anti-idiotype antibody specific for anti-Lewis Y monoclonal antibody. A further aspect of the invention is to provide a hybridoma, which is specific for anti-Lewis Y monoclonal antibody selected from the group consisting of LMH-1, LMH-2, and LMH-3.

As used herein, the term “humanized” antibody refers to a molecule that has its CDR's (complementarily determining regions) derived from a non-human species immunoglobulin and the remainder of the antibody molecule derived mainly from a human immunoglobulin.

The term “antibody” as used herein, unless indicated otherwise, is used broadly to refer to both antibody molecules and a variety of antibody derived molecules. Such antibody derived molecules comprise at least one variable region (either a heavy chain or light chain variable region) and include molecules such as Fab fragments, Fab′ fragments, F(ab′)₂ fragments, Fd fragments, Fabc fragments, Fd fragments, Fabc fragments, Sc antibodies (single chain antibodies), diabodies, individual antibody light chains, individual antibody heavy chains, chimeric fusions between antibody chains and other molecules, and the like.

The term “variable region” as used herein in reference to immunoglobulin molecules has the ordinary meaning given to the term by the person of ordinary skill in the act of immunology. Both antibody heavy chains and antibody light chains may be divided into a “variable region” and a “constant region”. The point of division between a variable region and a heavy region may readily be determined by the person of ordinary skill in the art by reference to standard texts describing antibody structure, e.g., Kabat et al “Sequences of Proteins of Immunological Interest: 5^{th} Edition” U.S. Department of Health and Human Services, U.S. Government Printing Office (1991).

The term “idiotype” as used herein refers to the segment of an antibody molecule that determines its specificity for antigen. The idiotype is located in the Fab region, and its expression usually requires participation of the variable regions of both heavy and light chains that form the antigen-combining site.

The term “isotype” as used herein refers to antigens that determine the class or subclass of heavy chains or the type and subtype of light chains of immunoglobulin molecules. For example, the four isotypes of IgG are designated IgG1, IgG2, IgG3, and IgG4.

The humanized anti-Lewis Y monoclonal antibody hu3S193 specifically targets the Lewis Y epithelial antigen. To assist the pharmacokinetic analysis of hu3S193 and determination of any immune responses to hu3S193 administered in the clinic, anti-idiotype hu3S193 antibodies were generated and characterized for suitability as ELISA reagents for measuring hu3S193 in patient sera samples and positive controls in HAHA analyses. Splenocytes from mice immunized with hu3S193 were fused with SP2/0-AG14 plasmacytoma cells and anti-idiotype antibodies produced by the resulting hybridomas were selected through ELISA for specific binding to hu3S193 and competitive binding for Lewis Y antigen. Nine hybridomas were initially selected and three, designated LMH-1, -2 and -3, showed specific binding to hu3S193 and competitive binding with hu3S193 to bind Lewis Y antigen. The recognition of hu3S193 idiotope was specific as demonstrated by lack of cross-reactivity with anti-Lewis Y monoclonal antibody, BR55.2 and with other irrelevant human IgG, murine IgG, humanized, or chimeric monoclonal antibodies.

Mouse monoclonal anti-idiotypes to anti-Lewis Y monoclonal antibody, hu3S193 were generated and their specificities defined enabling selection of particular anti-idiotypes for assays to monitor the pharmacokinetics and immune responses to hu3S193 administered in the clinic.

Three anti-idiotype hu3S193 antibodies, designated LMH-1, -2 and -3, were generated that could specifically bind hu3S193 and inhibit binding of hu3S193 and mu3S193, but not BR55.2 to Lewis Y antigen. The anti-hulgG mAb LMH-4 was also isolated and demonstrated strong binding activity to all tested chimeric humanized m Abs or purified hulgG. All four Abs were identified as murine IgG1 kappa isotype. The hybridoma LMH-3 produces 13 μg purified LMH-3 per ml culture supernatant in roller cultures, LMH-4 produces 15 μg/ml indicting these clones are amenable to large scale production. LMH-4 has potential as a reagent in the laboratory for the detection of hulgG or in the affinity purification of monoclonal antibodies from tissue culture supernatants contaminated with bovine IgG. BIAcore analyses determined a high apparent affinity of LMH-3 for hu3s193 of Ka=4.76×10⁸ M⁻¹ (Kd=2.10×10⁻⁹ M). This affinity enabled the development of a highly reproducible, sensitive, specific ELISA assay for determining serum concentrations of hu3S193 using purified LMH-3 for capture and biotinylated LMH-3 for detection. Establishment of HAHA analysis of sera samples by BIAcore was possible using LMH-3 as positive controls for quantitation of immune responses in patient sera and these anti-idiotypes could also be used to study the penetrance and binding of hu3S193 to tumor cells through immunohistochemical analysis of tumor biopsies. The generation of anti-idiotype antibodies capable of concurrently binding a target antibody on each variable domain provides reagents with high sensitivity for the assessment of safety and pharmacokinetic profiles of target antibodies administered clinically. Anti-idiotype hu3S193 mAbs have been generated which have significant use as diagnostic laboratory reagents for studying clinical samples from patients receiving hu3S193 immunotherapy.

Another aspect of the invention provides methods for detecting the ability of an anti-idiotype antibody to inhibit the binding of antibody to antigen. Another aspect of the invention is to provide methods for detecting the ability of anti-idiotype to capture and detect bound idiotype antibody. Another aspect of the invention is to provide methods for detecting the ability of anti-idiotype antibody to bind to idiotype anti-Lewis Y antibody. Another aspect of the invention is methods of detecting the amount of antibody in sample serum. The present invention is also directed against a method to detect HAMA, HACA and HAHA responses using the antibody of the invention

Two ELISA assays for determining serum hu3S193 levels were investigated. The first assay involved capture with synthetic Lewis Y antigen; the second employed anti-idiotype antibodies. The sensitivity and specificity of the assays were compared and analyses using LMH-3 were found superior due to a higher affinity for hu3S193 than the synthetic antigen, low background from lack of non-specific binding enabling high sensitivity and a serum hu3S193 limit of quantitation of 3 ng/ml. BIAcore analyses determined an apparent binding affinity of LMH-3 for hu3S193 of Ka=4.76×10⁸ M⁻¹ (Kd=2.10×10⁻⁹ M). The hybridoma produces high levels of antibody LMH-3.

The following examples are offered by way of illustration of the invention, and should not be interpreted as a limitation of the invention.

### EXAMPLES

### Example 1

### Cell Culture

All analytical grade reagents were purchased from Sigma Chemical Co. (St Louis, Mo., USA) unless otherwise indicated.

The SP2/0-Ag14 plasmacytoma cell line was purchased from American Type Culture Collection (ATCC; Manassas, Va., USA). The SP2/0 and selected hybridoma clones were cultured in Normal RPMI-1640 medium: RPMI-1640 medium (Gibco Invitrogen Corp., Mt Waverley, Victoria, Australia) containing 10% heat-inactivated fetal calf serum (FCS, TRACE Biosciences Pty Ltd, Sydney, Australia), 100 μg/ml streptomycin, 100 U/ml penicillin (Gibco) and 4 mM L-glutamine The initial few passages of fusion products were cultured with RPMI-1640 containing 20% FCS, penicillin, streptomycin, and L-glutamine as above. HAT medium was used for hybridoma selection; RPMI-1640 medium containing 20% FCS, HAT (hypoxanthine, aminopterin, thymidine), OPI (oxaloacetate, pyruvate, insulin media supplement) (Sigma), penicillin, streptomycin, L-glutamine. For hybridoma expansion, cells were cultured in HT medium: RPMI-1640 medium containing 20% FCS, HT (hypoxanthine, thymidine), penicillin, streptomycin, L-glutamine.

### Example 2

### Antibodies for Immunization and Screening

Humanized anti-Lewis Y IgG1 antibody hu3S193 (Lot: D01097, 10 mg/ml PBS), murine 3S193 (mu3S193), control isotype matched humanized mAb, huA33 (Lot: 5GMA33 10 mg/ml) and irrelevant isotype matched mouse: human chimeric IgG1 antibodies were provided by the Biological Production Facility, Ludwig Institute for Cancer Research, Melbourne, Australia. Purified human IgG was purchased from Sigma Chemical Co. F(ab)′2 fragments were prepared by digestion with immobilized pepsin according to the manufacturer's instructions (Pierce, Rockford, Ill., USA) and purified from undigested hu3S193 and Fc-containing fragments by passage over a 1 ml Protein-A column (Pharmacia Biotech, Uppsala, Sweden). The purified fragment was concentrated to 1 mg/ml on a Millipore centrifugal filter (Biomax 10k membrane, Millipore, Sydney, Australia).

### Example 3

### Immunization Protocol

Six to eight week old female BALB/c mice were purchased from the Breeding Facility, Walter and Eliza Hall Institute, Melbourne, Australia and housed with food and water provided ad libitum. All procedures performed with the animals for this study were approved by the Animal Ethics Committee of the Austin and Repatriation Medical Centre. Pre-immunization venous blood samples (200 μl) were collected from each animal and coagulated at RT for 1 h and 4° C. for overnight. The resulting serum (˜100 μl/mouse) was collected and stored at −20° C.

The immunization procedure was performed with six mice. On Day 0 BALB/c mice were immunized by i.p. injection of 200 μl containing 40 μg purified hu3S193: complete Freund's adjuvant (1:1, v:v). Mice received booster injections on Days 14 and 28 containing 40-μg hu3S193 in incomplete Freund's adjuvant. On Day 35, a post-immunization blood sample was collected from the tail vein and the serum titer of mouse anti-hu3S193 antibody determined by ELISA. Pre-immunization serum samples were included as controls. On three occasions a mouse was sacrificed 4 days after a final i.v. booster injection containing 40 μg hu3S193 alone. The spleens were aseptically collected into 5 ml PBS/G, and the splenocyte suspension were prepared for fusion.

### Example 4

### Fusion and Hybridoma Generation

All procedures were performed at room temperature and were based upon previously published methods (12). Splenocytes were collected and washed three times with RPMI-1640 medium and the cell concentration determined. Simultaneously the fusion partner SP2/0 cells were harvested and washed in serum-free media and counted. Each cell pellet was resuspended in 5 ml serum-free RPMI and then combined at a splenocytes: SP2/0 ratio of 5:1 in a 50 ml tube. The cell mixture was centrifuged, 300×g, 10 min and the resulting cell pellet disrupted gently by tapping. The tube was incubated in a 37° C. water bath and 50% PEG in medium (1 ml) was added drop-wise over 1 min. Pre-warmed serum-free RPMI (2 ml) was then added over 2 min, followed by 8 ml medium over 3 min. The cells were centrifuged, 300×g 10 min, and re-suspended gently in HAT medium (60 ml HAT medium/mouse spleen containing ˜1×10 5 splenocytes, 2×10 4 SP2/0 myeloma cells). The mixed cell suspension was aliquoted (100 μl/well) into six 96-well tissue culture plates (Falcon; Becton Dickinson, Franklin Lakes, N.J., USA) and cultured at 37° C. in a humidified 5% CO₂/air incubator.

### Example 5

### Monoclonal Antibody Selection

Hybridoma culture supernatants were screened by ELISA for positive binding activity with hu3S193 mAb and purified human IgG. Selected clones were expanded in 24-well culture plates (Falcon) and their binding activity and competitive binding activities were further characterized by ELISAs. The subclass of the selected anti-idiotype hu3S193 antibodies in culture supernatants was determined with the mouse monoclonal antibody isotyping kit (IsoStrip, Roche Diagnostics GmbH, Mannheim, Germany).

### Example 6

### Cloning Hybridomas by Limiting Dilution

Primary dilution series. The selected hybridoma clones were plated at a density of 100 cells/well in 200-μl HAT medium in the inner 60 wells of the 96 well plate. The outer wells were filled with RPMI-1640 medium containing antibiotics to prevent dehydration and concentration of the HAT medium/cell containing wells. When cell growth was almost confluent (˜7 days) and the supernatants were collected and tested for specific binding to hu3S193 and antagonist binding to hu3S193 for Lewis Y antigen binding. Positive clones were selected for continued analyses and cloning.

Secondary dilution series. This step aimed to establish hybridomas grown from a single cell and the culturing utilized a splenocyte feeder layer. The splenocytes were prepared from non-immunized BALB/c mice, resuspended at 10 6 cells/ml HAT medium supplemented with 100 mM 2-mercaptoethanol, and incubated overnight at room temperature in a sterile environment to ensure no contaminant infection. The following day two 96-well microtitre plates were prepared for each selected clone with only the inner 60 wells of the plate used, as previously described. Aliquots (100 μl) of HAT medium were added to Row 1, the splenocyte suspension was added to Rows 2-6 (10 5 cells/well), and the plates incubated at 37° C. overnight. The selected cells were diluted in HAT medium and 100 μl aliquots containing 100, 10, 5, 1, or 0.5 cells added to wells of rows 2 through 6, respectively, containing feeder cells. Plates were incubated at 37° C. for 10-14 days and clonal colonies were selected from the wells seeded with 1 or 0.5 cells/well.

Third dilution series. To ensure that the selected clones were derived from a single cell, all selected cell lines underwent a repeat round of the second-dilution procedure. The preferred lines were expanded and stocks prepared and stored under liquid nitrogen.

### Example 7

### Antibody Production

Selected and re-cloned anti-idiotype antibody producing hybridomas were cultured in roller bottles (Corning, Corning Incorporated, NY, USA) and the culture supernatant harvested aseptically. Anti-idiotype antibodies were purified by affinity chromatography on a 5 ml protein A sepharose fast flow column (Amersham Pharmacia Biotech, Uppsala, Sweden) pre-equilibrated in 50 mM Tris-HCl, pH 8 (buffer A). Following washing with 20 column volumes buffer A, bound antibody was eluted using 100 mM Glycine-HCL, pH 2.7 containing 200 mM NaCl and immediately neutralized using 1M Tris HCl, pH 8. Following overnight dialysis at 4° C. into PBS, the antibody was concentrated with a centrifugal filter (Millipore, Biomax 10k membrane). The purity was examined by SDS-PAGE under reducing and non-reducing conditions. Protein was visualized by Coomassie Blue staining. Biotinylated anti-idiotype antibodies were prepared using the ECL protein biotinylation module kit according to the manufacturer's instructions (Amersham Pharmacia Biotech.).

### Example 8

### ELISA Analysis for Binding Specificity

The binding specificities of hybridoma supernatants or purified anti-idiotype antibodies were characterized by ELISA analysis. The 96-well plates (Nunc MaxiSorp, Roskilde Denmark) were coated with hu3S193 (100 μl, 4.0 μg/ml), purified human IgG, or other control monoclonal antibodies in carbonate buffer (0.05 M, pH 9.6) overnight at 4° C. Non-specific binding was blocked with 3% BSA/PBS at room temperature (23° C.) for 2 h. Serial dilutions of supernatants or purified antibodies (1:2-1:6300) were prepared, and 100 μl aliquots added per well. Positive (hulgG) and negative (conjugate and substrate alone) controls were included with each assay. Plates were incubated 1 h at room temperature, washed three times with 0.05% Tween 20/PBS then incubated for 1 h with secondary antibody (goat anti-mouse IgG-alkaline phosphatase conjugated (100 μl/well, 1:3000 dilution in 1% BSA/PBS). Bound antibody was detected with chromophore substrate p-Nitrophenyl phosphate substrate (ICN Biomedicals Inc., Aurora, Ohio, USA). The optical density was measured at 450 nm with a Vmax kinetic microplate reader (Molecular Devices Corporation, Sunyvale, Calif.).

### Example 9

### ELISA Analysis for Antagonist Activity

Selected anti-idiotype antibodies were tested for their ability to block the binding of hu3S193 mAb to the Lewis Y-antigen coated plates. Synthetic Lewis Y tetrasaccharide is available coupled to BSA at a molar ratio of ˜32:1 (Alberta Research Council, Edmonton, Alberta, Canada). ELISA plates (Nunc MaxiSorp) were coated with Lewis Y-BSA antigen (50 μl 3.0 μg/ml PBS and incubated at 4° C. overnight. Plates were blocked for non-specific binding with 3% BSA/PBS at room temperature for 2 h. Half-log serially diluted samples (supernatant or purified anti-idiotype mAbs) were added to each well followed by hu3S193 mAb (50 μl; 1 μg/ml final concentration). Following incubation at room temperature for 1 h, goat anti-human IgG-HRP was used to detect bound hu3S193 mAb and after extensive washings visualized with ABTS substrate (2,2′-azino-bis-(3-ethyl-benzthiazoline-6-sulfonic acid; 100 μl 40 μM/well; ICN). The optical density (OD) was read at 415 nm using Vmax kinetic microplate reader.

### Example 10

### Anti-Idiotype Specificity for Hu3S193

The specificity of purified selected clones for binding to anti-Lewis Y mAbs was further examined using hu3S193, and murine mAbs mu3S193 and BR55.2 (Ludwig Institute for Cancer Research, New York) hu3S193. The specificity was examined with two ELISAs. The first utilized the anti-idiotype for capture and detection of bound idiotype mAb and the second examined the ability of the anti-idiotype mAb to bind idiotype anti-Lewis Y mAbs in solution and inhibit Lewis Y antigen binding.

For the first assay an ELISA Maxisorp plate was coated with selected anti-idiotype mAb overnight at 4° C. (100 μl 4 μg/ml 0.05M carbonate buffer, pH 9.6). Following blocking with 3% FCS/PBS, serially diluted triplicate samples of 10 μg/ml hu3S193, mu3S193, BR55.2 and control irrelevant mAbs huA33 and muA33 were added to the wells then incubated at room temperature, 1 hr. After washing, bound mAb was detected with biotinylated anti-idiotype mAb (LMH-3-Bt; 100 μl/well 4 μg/ml in 1% FCS/PBS at 4° C., overnight). For detection of bound complexes strepavidin-HRP (1:1000 dilution in 1% FCS/PBS, 100 μl/well) followed by ABTS for color development were added. OD415 was measured as previously described.

For the second test of specificity three ELISA plates were coated with synthetic Lewis Y-BSA antigen (4.0 μg/ml in PBS, overnight at 4° C.). Serially diluted anti-Lewis Y mAbs were then aliquoted across each row of the plate; plate 1, hu3S193; plate 2, mu3S193; plate 3 BR55.2 final concentration range (0.003-10-μg/ml). To test competition for binding to Lewis Y antigen, the other two anti-Lewis Y mAbs and two anti-idiotype mAbs were added in duplicate (10 μg/ml final concentration), then incubated at room temperature for 1 h. Following washing, bound hu3S193 in plate 1 was detected with anti-hulgG-HRP (Sigma; 100 μl 1:1000 in 1% FCS/PBS) followed by ABTS for color development and A415 nm measurement. Bound mu3S193 on plate 2 and BR55.2 on plate 3 were detected with goat anti-mIgG-alkaline phosphatase and p-nitrophenol chromogen measured at A405.

### Example 11

### ELISA Measurement of Hu3S193 in Serum

Two ELISA assays were developed for testing hu3S193 mAb in serum samples. For the first assay synthetic Lewis Y-BSA antigen (3.0 μg/ml in PBS, 50 μl/well) was utilized for hu3S193 capture. Following incubation with serially diluted samples of hu3S193 in healthy donor serum or 3% BSA/PBS, the amount of hu3S193 mAb binding to antigen was detected with goat anti-human IgG-HRP secondary antibody and ABTS substrate.

The second assay involved coating with anti-idiotype antibody and detection with biotinylated anti-idiotype antibodies. Plates were coated with anti-idiotype hu3S193 antibody (3.0 μg/ml in 0.05M carbonate buffer pH 9.6, 100 μl/well) overnight at 4° C., then blocked with 3% BSA/PBS for 2 h at room temperature (23° C.). Serially diluted samples of hu3S193 (0.0315-10 μg/ml) in 3% BSA/PBS or healthy donor serum were added to the plates in triplicate, and incubated 1 h at room temperature. Following washing, biotinylated anti-idiotype hu3S193 antibody was added (100 μl 3 μg/ml in 1% BSA/PBS), and incubated 1 h room temperature. For detection of bound complexes strepavidin-HRP (1:1000 dilution in 1% BSA/PBS, 100 μl/well) followed by ABTS for color development were added. OD415 was measured as previously described.

Biosensor Analysis Biosensor analyses were performed using a BIAcore 2000 (BIAcore AB, Uppsala, Sweden) on a carboxymethyldextran coated sensor chip (CM5). The chip was derivatized with hu3S193 on channel 2, LMH-3 on channel 3 and synthetic Lewis y tetrasaccharide, coupled to bovine serum albumin (Alberta Research Council, Edmonton, Canada) on channel 4, using standard NHS/EDC amine coupling chemistry. Samples of hu3S193 and LMH-3 were diluted in HBS buffer (10 mM HEPES, pH 7.4; 150 mM NaCl; 3.4 mM di-Na-EDTA; 0.005% Tween-20), and aliquots (30 μl) were injected over the sensor chip surface at a flow rate of 5 μl/min. After the injection phase, dissociation was monitored by flowing HBS buffer over the chip surface for 300s. Bound antibody was eluted and the chip surface regenerated between samples by injection of 20 μl of 100 mM glycine/100 mM NaCL, pH 2.7. For kinetic analyses of binding, varying concentrations of hu3S193 and LMH-3 were injected over the sensor chip surfaces. Apparent association (ka) and dissociation (kd) rate constants were calculated using a bivalent analyte model, with global, and local fitting for calculation of Rmax, using BIAevaluation v3.0 software (Pharmacia Biosensor, Uppsala, Sweden).

Patient immune responses to administered monoclonal antibodies can be assessed by Biocore® analysis of patient sera (Ritter, Cohen et al. 2001). Anti-idiotype antibodies are useful as positive control reagents in these analyses. Accordingly, LMH-3 was added to healthy donor sera (3-50 μg/ml) and binding responses to immobilized hu3S193 were validated.

### Example 12

### Mice Immunization and Hybridoma Clone Selection

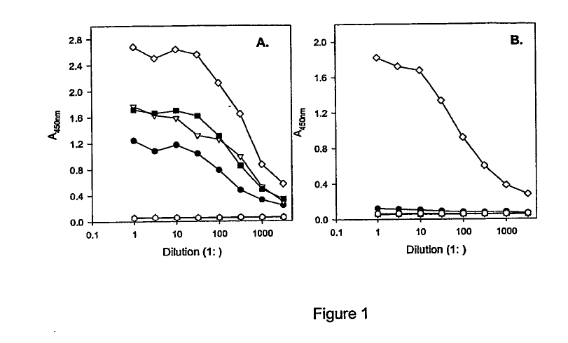

Three groups of BALB/c mice were immunized repeatedly with hu3S193 mAb, and their sera assayed for mouse anti-hu3S193 mAb activity. Immunoreactivity of pre- and post-immunization sera samples indicated the development of high titer mouse anti-intact and F(ab)′2 hu3S193 mAbs. Plates coated with an isotype control humanized IgG (huA33) or chimeric IgG F(ab)′2 also showed strong positive binding (results not shown). The mice were sacrificed and their spleens removed. The splenocytes and partner fusion Sp2/0 plasmacytoma cells were fused and hybridomas from these three fusions were screened. Only 34 wells demonstrating strong binding activity for hu3S193 mAb but weak or no binding to control huA33 or purified human IgG were selected and expanded. After expansion, the antibody specificity was further tested with ELISA assay (FIG. 1). Three control humanized or chimeric antibodies were used during the selection of clones from the first fusion. Because cross-binding activity was usually observed with purified human IgG coated plates, in the subsequent screenings purified human IgG was primarily used in addition to hu3S193.

### Example 13

### Binding and Blocking Activities of Selected Anti-Idiotype Antibodies

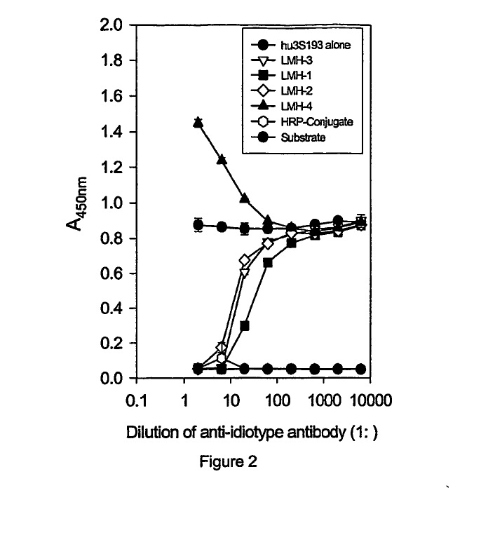

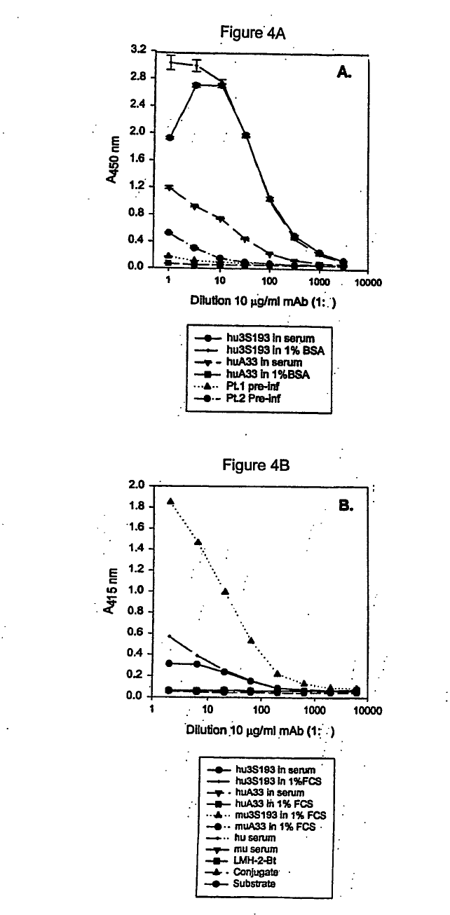

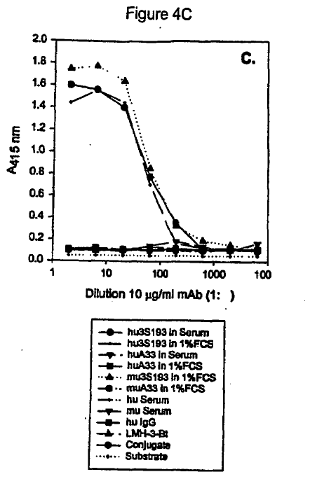

The binding and antagonistic activities of all selected hybridomas were further analyzed to identity hybridomas producing anti-idiotype hu3S193 antibody. Half-log serial dilutions were prepared from each hybridoma culture supernatant and evaluated by ELISA. Of the 12 clones initially selected, three were identified that could bind hu3S193, but not hulgG and inhibit hu3S193 binding to Lewis Y antigen (FIG. 1). These hybridomas, F1-1, F2-3 and F3-27, were cloned from single cells by limiting dilution and designated Ludwig Institute for Cancer Research Melbourne Hybridoma (LMH)-1, LMH-2 and LMH-3, respectively. One of the clones (FS2-1) demonstrated strong anti-hulgG binding activity to all tested chimeric humanized mAbs or purified hulgG or (FIG. 1) and was subsequently used as a positive control for hulgG binding. This clone was prepared from a single cell and named LMH-4. LMH-1 through -4 antibodies were identified as isotype IgG1 ê by mouse monoclonal antibody isotyping kit and were purified from culture supernatants by protein-A affinity chromatography. The four clone's binding specificities were confirmed by ELISA (FIG. 4A-C). No cross-reactivity with hulgG constant chains were observed for LMH-1, -2 or -3 (FIG. 4B) and all three antibodies specifically bound hu3S193 mAb alone (FIG. 4A). The antagonistic activities of these antibodies were analyzed by a competitive ELISA assay. LMH-1, -2 and—3 anti-hu3S193 idiotype antibodies bound hu3S193 mAb and inhibit binding of hu3S193 mAb to Lewis Y antigen in a dose dependent manner (FIG. 2).

### Example 14

### Determination of Antibody Binding Affinity

The apparent binding affinity for the Biosensor binding experiments of immobilized hu3S193 interacting with LMH-3 in solution determined a Ka of 4.76×10⁸ M⁻¹ (Kd=2.10×10⁻⁹ M). In the reciprocal experiment with LMH-3 immobilized on the chip and running hu3S193 over the surface as the analyte, a Ka of 9.12×10⁸ M⁻¹ (Kd=1.10×10⁻⁹ M was determined.

### Example 15

### Anti-Idiotype Specificity for Hu3S193

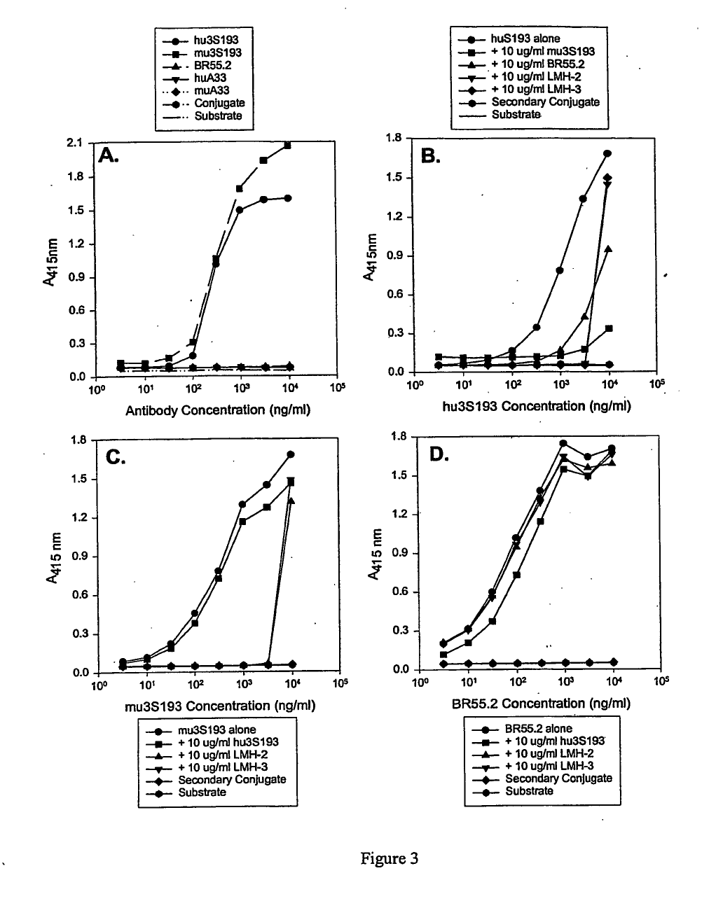

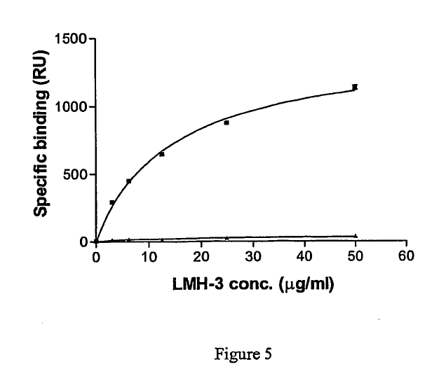

The specificity of clones LMH-2 and LMH-3 for the 3S193 idiotype was verified in these experiments (FIG. 5.) Protein-A purified LMH-3 was coated on ELISA plates and serially diluted triplicate samples of 10 μg/ml hu3S193, mu3S193, BR55.2 and control IgG1 mAbs huA33 and muA33 were added to the wells to further characterize the binding specificity of LMH-3. An additional favorable feature of this clone was that biotinylated LMH-3 could be utilized for detection of bound 3S193 mAb captured with LMH-3. The ELISA results (FIG. 3A) indicate that LMH-3 specifically binds the anti-Lewis Y 3S193 idiotope, with no reactivity with the murine anti-Lewis Y BR55.2 detected, and no cross reactivity with murine and human IgG1 constant domains of control muA33 and huA33. The binding of hu3S193 and mu3S193 to Lewis Y antigen was effectively competed for in solution by 10 μg/ml LMH-2 and LMH-3, but upon saturation of the anti-idiotype binding sites, some hu3S193 and mu3S193 binding to antigen was observed (FIGS. 3B and 3C, respectively). The murine BR55.2 mAb effected some competitive binding while the higher affinity mu3S193 completely blocked hu3S193 binding to Lewis Y antigen except at the highest concentration (10 μg/ml) (FIG. 3B). In the reciprocal experiment of plate 2, the lower affinity hu3S193 could not compete for Lewis Y antigen when mu3S193 was added first to the wells (FIG. 3C). The results from plate 3 (FIG. 3D) demonstrate that BR55.2 binding to Lewis Y antigen could not be competed for or inhibited by hu3S193 or the anti-idiotype clones LMH-2 and -3. Thereby confirming the specificity of these clones for the 3S193 idiotope. The competition by BR55.2 observed in FIG. 3B may be achieved through steric hindrance upon high affinity binding to its different Lewis Y epitope.

### Example 16

### Development of ELISA Assay for Measuring Hu3S193 in Clinical Samples

The first ELISA assay investigated employed synthetic antigen (Lewis Y tetrasaccharide-BSA conjugate) for capture of hu3S193 in serum and secondary goat anti-human IgG conjugated with peroxidase for detection. The synthetic Lewis Y antigen has a comparatively lower affinity for the hu3S193 mAb that reduced the sensitivity of the assay as indicated by the low optical density values recorded. The secondary conjugated antibody detects all human immunoglobulins and accordingly high background was observed for all samples analyzed (FIG. 4A.). The high background interference markedly reduces the sensitivity and accuracy of the assay, particularly for low serum hu3S193 concentrations. The second ELISA assay used purified anti-idiotype hu3S193 antibodies LMH-1, 2, and -3 for coating, biotinylated LMH-1, -2 or -3 as secondary antibody, and strepavidin-HRP and ABTS substrate for visualization of bound complexes. LMH-1 and LMH-2 anti-idiotype antibodies showed strong binding for mu3S193 mAb in serum or in 3% BSA/PBS, but weak binding for hu3S193 mAb. However no cross-binding activity was observed with control antibodies, including humanized mAb, chimeric mAb, murine mAb, Ig in human serum or Ig in mouse serum (FIG. 4B). Attempts to optimize or improve the ELISAs with LMH-1 and LMH-2 by varying the concentration of coating antibody or secondary antibody and using different coating buffers were investigated. Some improvements were achieved, but no change to the response pattern was effected (data not shown). However using LMH-3 anti-idiotype mAb for capture and LMH-3-Biotin as secondary antibody, specific, strong binding was observed for both hu3S193 and mu3S193 mAbs in serum with minimal non-specific background activity (FIG. 4C). The highly reproducible assay demonstrated excellent sensitivity and the limit of detection for hu3S193 or mu3S193 mAbs in serum samples was 3 ng/ml.

### BIOCore Analyses of Immune Responses

The measurement of HAHA in serum was developed for hu3S193 using anti-idiotype LMH-3. A sensitive and reproducible method was established and validated (FIG. 5).

### REFERENCES

Bhattacharya-Chatterjee, M., S. K. Chatterjee, et al. (2001). “The anti-idiotype vaccines for immunotherapy.” Curr Opin Mol Ther 3(1): 63-9.
Brown, G. and N. Ling (1988). Murine Monoclonal Antibodies. Antibodies, Volume I, A Practical Approach. D. Catty. Oxford, England, IRL Press: 81-104.
Clark, M. (2000). “Antibody humanization: a case of the ‘Emperor's new clothes’?” Immunol Today 21(8): 397-402.
Clarke, K., F. T. Lee, et al. (2000a). “In vivo biodistribution of a humanized anti-Lewis Y monoclonal antibody (hu3S193) in MCF-7 xenografted BALB/c nude mice.” Cancer Res 60(17): 4804-11.
Clarke, K., F. T. Lee, et al. (2000b). “Therapeutic efficacy of anti-Lewis(y) humanized 3S193 radioimmunotherapy in a breast cancer model: enhanced activity when combined with taxol chemotherapy.” Clin Cancer Res 6(9): 3621-8.
Fields, B. A., F. A. Goldbaum, et al. (1995). “Molecular basis of antigen mimicry by an anti-idiotope.” Nature 374(6524): 739-42.
Glennie, M. J. and P. W. Johnson (2000). “Clinical trials of antibody therapy.” Immunol Today 21(8): 403-10.
Gruber, R., L. J. van Haarlem, et al. (2000). “The human antimouse immunoglobulin response and the anti-idiotypic network have no influence on clinical outcome in patients with minimal residual colorectal cancer treated with monoclonal antibody CO17-1A.” Cancer Res 60(7): 1921-6.
Hoffman, E. W., A. M. Scott, et al. (2001). Phase I trials of CDR-grafted humanized monoclonal antibody hu3S193 in patients with Lewis-Y expressing solid tumors. ASCO 37th Annual Meeting, San Francisco.
Jerne, N. K. (1974). “Towards a network theory of the immune system.” Ann Immunol (Paris) 125C(1-2): 373-89.
Kim, Y. S., M. Yuan, et al. (1986). “Expression of LeY and extended LeY blood group-related antigens in human malignant, premalignant, and nonmalignant colonic tissues.” Cancer Res 46(11): 5985-92.
Kitamura, K., E. Stockert, et al. (1994). “Specificity analysis of blood group Lewis-y (Le(y)) antibodies generatedagainst synthetic and natural Le(y) determinants.” Proc Natl Acad Sci USA 91(26): 12957-61.
Liu, Z., F. E. Smyth, et al. (2002). “Anti-renal cell carcinoma chimeric antibody G250: cytokine enhancement of in vitro antibody-dependent cellular cytotoxicity.” Cancer Immunol Immunother 51(3): 171-7.
Ritter, G., L. S. Cohen, et al. (2001). “Serological analysis of human anti-human antibody responses in colon cancer patients treated with repeated doses of humanized monoclonal antibody A33.” Cancer Res 61(18): 6851-9.
Safa, M. M. and K. A. Foon (2001). “Adjuvant immunotherapy for melanoma and colorectal cancers.” Semin Oncol 28(1): 68-92.
Sakamoto, J., K. Furukawa, et al. (1986). “Expression of Lewisa, Lewisb, X, and Y blood group antigens in human colonic tumors and normal tissue and in human tumor-derived cell lines.” Cancer Res 46(3): 1553-61.
Scott, A. M., D. Geleick, et al. (2000). “Construction, production, and characterization of humanized anti-Lewis Y monoclonal antibody 3S193 for targeted immunotherapy of solid tumors.” Cancer Res 60(12): 3254-61.
Steffens, M. G., O. C. Boerman, et al. (1997). “Targeting of renal cell carcinoma with iodine-131-labeled chimeric monoclonal antibody G250.” J Clin Oncol 15(4): 1529-37.
Uemura, H., E. Okajima, et al. (1994). “Internal image anti-idiotype antibodies related to renal-cell carcinoma-associated antigen G250.” Int J Cancer 56(4): 609-14.
Zhang, S., C. Cordon-Cardo, et al. (1997). “Selection of tumor antigens as targets for immune attack using immunohistochemistry: I. Focus on gangliosides.” Int J Cancer 73(1): 42-9.

We claim:

1. An anti-idiotype antibody directed against Anti-Lewis Y monoclonal antibody.

2. The anti-idiotype antibody of claim 1, which binds to the variable region of an anti-Lewis Y monoclonal antibody.

3. The anti-idiotype antibody of claim 1, which blocks the binding of an anti-Lewis Y monoclonal antibody.

4. The anti-idiotype antibody of claim 1 which specifically binds hu3S193.

5. The anti-idiotype antibody of claim 1 which is selected from the group consisting of a monoclonal antibody, a chimeric antibody, a human antibody, a humanized antibody, or a single chain antibody.

6. The anti-idiotype antibody of claim 2 which is selected from the group consisting of a monoclonal antibody, a chimeric antibody, a human antibody, a humanized antibody, or a single chain antibody.

7. The anti-idiotype antibody of claim 3 which is selected from the group consisting of a monoclonal antibody, a chimeric antibody, a human antibody, a humanized antibody, or a single chain antibody.

8. The anti-idiotype antibody of claim 4 which is selected from the group consisting of a monoclonal antibody, a chimeric antibody, a human antibody, a humanized antibody, or a single chain antibody.

9. A hybridoma capable of producing the anti-idiotype antibody of claim 1.

10. A hybridoma capable of producing the anti-idiotype antibody of claim 2.

11. A hybridoma capable of producing the anti-idiotype antibody of claim 3.

12. A hybridoma capable of producing the anti-idiotype antibody of claim 4.

13. The hybridoma of claim 12, which is, selected from the group consisting of LMH-1, LMH-2, and LMH-3.

14. The anti-idiotype antibody of claim 4, which is produced by a hybridoma, selected from the group consisting of LMH-1, LMH-2, and LMH-3.

15. A method of detecting binding specificity of anti-idiotype antibodies comprising:
a. coating an Elisa plate with anti-Lewis Y antibody, purified human IgG or other control Mab;
b. adding purified anti-idiotype antibody;
c. incubating with secondary antibody; and
d. detecting the amount of bound anti-idiotype antibody, wherein binding of an anti-idiotype antibody to the antibody evidences binding specificity.

16. The method of claim 15 wherein the mAB is hu3S193.

17. The method of claim 15 wherein the anti-idiotype antibody is directed against anti-Lewis Y antigen.

18. A method of detecting anti-idiotype anti-Lewis Y antibodies capable of blocking the binding of anti-Lewis Y monoclonal antibody comprising:
a. coating an Elisa plate with Lewis Y-BSA coupled antigen;
b. adding anti-idiotype antibody;
c. adding monoclonal anti-Lewis Y idiotype antibody; and
d. detecting bound anti-Lewis Y monoclonal antibody, wherein the amount of anti-Lewis Y antibody bound to the antigen in the presence and absence of anti-idiotype antibody is evidence of the ability of the anti-idiotype antibody to block the binding of anti-Lewis monoclonal antibody.

19. The method of claim 18 wherein the anti-anti-Lewis Y monoclonal antibody is hu3S193.

20. The method of claim 18 wherein the anti-idiotype antibody is directed against anti-Lewis Y antibody

21. A method of detecting anti-Lewis Y antibody in a serum sample comprising:
a. coating ELISA plate with synthetic Lewis Y-BSA antigen;
b. adding serum samples to the ELISA plates;
c. adding peroxidase conjugated anti-Lewis Y anti-idiotype antibody to the ELISA plates; and
d. determining the presence of anti-Lewis Y antibody from the amount of peroxidase conjugated anti-Lewis Y anti-idiotype antibody bound to antigen-coated ELISA plate.

22. The method of claim 21 wherein the monoclonal idiotype antibody is hu3S193

23. A method of detecting an anti-Lewis Y HAHA response in a subject who has been administered a humanized anti-Lewis Y monoclonal antibody comprising:
a. collecting serum sample from the subject;
b. reacting the serum sample to a Lewis Y antigen coated ELISA plate in the presence or absence of a peroxidase conjugated anti-Lewis Y antibody; and
c. determining there from the presence or absence of anti-idiotype anti-Lewis antibody, wherein presence of anti-idiotype anti-Lewis antibody evidences a HAHA response.
